(19) United States
(12) Patent Application Publication
(10) Pub. No.: US 2008/0027284 A1
(43) Pub. Date: Jan. 31, 2008

(54) Endoscope Having Power Shutdown Unit

(75) Inventor: Tadaaki SUDA, Saitama (JP)

Correspondence Address: GREENBLUM & BERNSTEIN, P.L.C., 1950 ROLAND CLARKE PLACE, RESTON, VA, 20191, US

(73) Assignee: PENTAX CORPORATION, Tokyo (JP)

(21) Appl. No.: 11/782,730

(22) Filed: Jul. 25, 2007

(30) Foreign Application Priority Data
Jul. 28, 2006 (JP) 2006-205705

(51) Int. Cl. A61B 1/00

(52) U.S. Cl. 600/134

(57) ABSTRACT

An endoscope to obtain an image inside a body cavity is provided. The endoscope includes an image capturing element, a power source, which supplies electric power to the image capturing element, a power shutdown unit, which shuts down the electric power to the image capturing element so that the image capturing element can be prevented from overcurrent, a drive control unit, which outputs control signals to the image capturing element in order to control driving of the image capturing element based on driving signals provided from an external unit connected with the endoscope, and a control restricting unit, which restricts the drive control unit to cease outputting the control signals to the image capturing element in response to activation of the power shutdown unit.

### BACKGROUND OF THE INVENTION

The present invention relates to an electronic endoscope with an image capturing element and an endoscope system having the endoscope and signal processor to process images captured by the image capturing element so that the processed image can be displayed on a monitor to be viewed.

Conventionally, an endoscope system having an electronic endoscope with an image capturing element in a front end unit thereof and a processor to process signals obtained through the image capturing element and output the processed signals to a monitor is widely used. The front end unit of the electronic endoscope is designed to be smaller in a length and a diameter thereof in consideration of burden on a patient (subject). A drive circuit, for example, to drive the image capturing element is arranged in a proximal end portion of the endoscope rather than in the front end unit so that a size of the front end unit can be minimized. The drive unit and the image capturing element are connected to each other with a signal cable arranged inside a flexible tube of the endoscope, for example, by soldering.

When the flexible tube of such an endoscope is bent during use, the signal cable inside the flexible tube is bent accordingly. As such a bending operation is repeatedly performed, repeated stress is applied to the connecting portion between the image capturing element and the signal cable, and excessive stress may cause disconnection of the cable from the image capturing element at the connecting portion. Further, external impact applied to the flexible tube may cause conflict and friction between the signal cable and the other component. The signal cable can be thus worn and damaged, and the internal cables can be exposed.

The disconnected signal cable and the exposed internal cables may cause short-circuiting in the endoscope system including the image capturing element and the drive circuit. Further, the short-circuiting may cause overcurrent in the components, and the components may be overheated.

In order to prevent the overheating in the components due to the short-circuiting, an electronic power unit having a power shutdown circuit is provided. An example of such power unit is disclosed in Japanese Patent Provisional Publication No. 2005-38281. In this power unit, when overcurrent is caused in the circuit, the electric current is shutdown by one of a fuse arranged at an upstream (input) side of the power unit and the an overcurrent detecting circuit arranged at a downstream (output) side of the power unit.

Thus, it is considered such an overcurrent detecting circuit may be applied in the endoscope system as described above so that the overheating in the components in the endoscope due to the short-circuiting can be prevented. However, even when the electric current supplied to the image capturing element is shutdown by the power shutdown circuit, the image capturing element remains receiving the driving signals from the driving circuit. Thus, considerably higher voltage than the power supply voltage (e.g., 0 volt when the electric current is shutdown) is applied to the image capturing element. In this situation, a latch-up phenomenon, in which the image capturing element can be overheated and fail, may be easily caused.

### SUMMARY OF THE INVENTION

In view of the foregoing drawbacks, the present invention is advantageous in that an electronic endoscope and an endoscope system, in which the latch-up phenomenon can be prevented, are provided.

According to an aspect of the present invention, there is provided an endoscope to obtain an image inside a body cavity. The endoscope includes an image capturing element, a power source, which supplies electric power to the image capturing element, a power shutdown unit, which shuts down the electric power to the image capturing element so that the image capturing element can be prevented from overcurrent, a drive control unit, which outputs control signals to the image capturing element in order to control driving of the image capturing element based on driving signals provided from an external unit connected with the endoscope, and a control restricting unit, which restricts the drive control unit to cease outputting the control signals to the image capturing element in response to activation of the power shutdown unit.

Optionally, the power shutdown unit may be a fuse provided between the power source and the image capturing element, and activating the power shutdown unit may be blowing the fuse.

Optionally, the endoscope may further include a fuse status judging system, which judges as to whether the fuse is blown. The control restricting unit may restrict the drive control unit to cease outputting the control signals to the image capturing element when the fuse status judging system judges that the fuse is blown.

According to another aspect of the present invention, there is provided an endoscope system. The endoscope system includes an endoscope to obtain an image inside a body cavity a monitor, which displays the image obtained by the endoscope, and a processor, which processes the image obtained by the endoscope into a format adaptable to the monitor. The endoscope includes an image capturing element, a power source, which supplies electric power to the image capturing element, a power shutdown unit, which shuts down the electric power to the image capturing element so that the image capturing element can be prevented from overcurrent, a drive control unit, which outputs control signals to the image capturing element in order to control driving of the image capturing element based on driving signals provided from an external unit connected with the endoscope, and a control restricting unit, which restricts the drive control unit to cease outputting the control signals to the image capturing element in response to activation of the power shutdown unit.

Optionally, the power shutdown unit of the endoscope may be a fuse provided between the power source and the image capturing element, and activating the power shutdown unit may be blowing the fuse.

Optionally, the endoscope may further include a fuse status judging system, which judges as to whether the fuse is blown. The control restricting unit of the endoscope may restrict the drive control unit to cease outputting the control signals to the image capturing element when the fuse status judging system judges that the fuse is blown.

Optionally, a reporting signal to report the activation of the power shutdown unit may be transmitted to the processor in response to the activation of the power shutdown unit; and the processor may add a superimposed image over the image obtained by the endoscope on the monitor in response to the reporting signal.

According to another aspect of the present invention, there is provided an electric power unit for an endoscope with an image capturing element to obtain an image inside a body cavity. The electronic power unit includes a power source, which supplies electric power to the image capturing element, a power shutdown unit, which shuts down the electric power to the image capturing element so that the image capturing element can be prevented from overcurrent, a drive control unit, which outputs control signals to the image capturing element in order to control driving of the image capturing element based on driving signals provided from an external unit connected with the endoscope, and a control restricting unit, which restricts the drive control unit to cease outputting the control signals to the image capturing element in response to activation of the power shutdown unit.

### BRIEF DESCRIPTION OF THE DRAWINGS

FIG. 1 is a schematic view of an endoscope system according to an embodiment of the present invention.

FIG. 2 is a block diagram illustrating a configuration of the endoscope system according to the embodiment of the present invention.

FIG. 3 is a block diagram illustrating a configuration of a CCD (charge-coupled device) and a DSP (digital signal processor) board of the endoscope system according to the embodiment of the present invention.

### DETAILED DESCRIPTION OF THE EMBODIMENTS

Hereinafter, referring to the accompanying drawings, according to illustrative embodiments of the invention will be described.

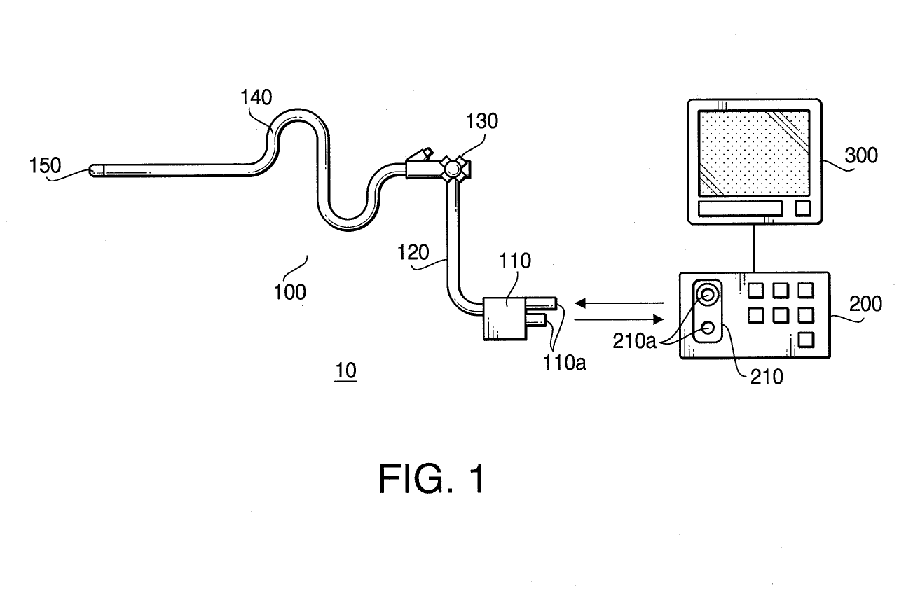

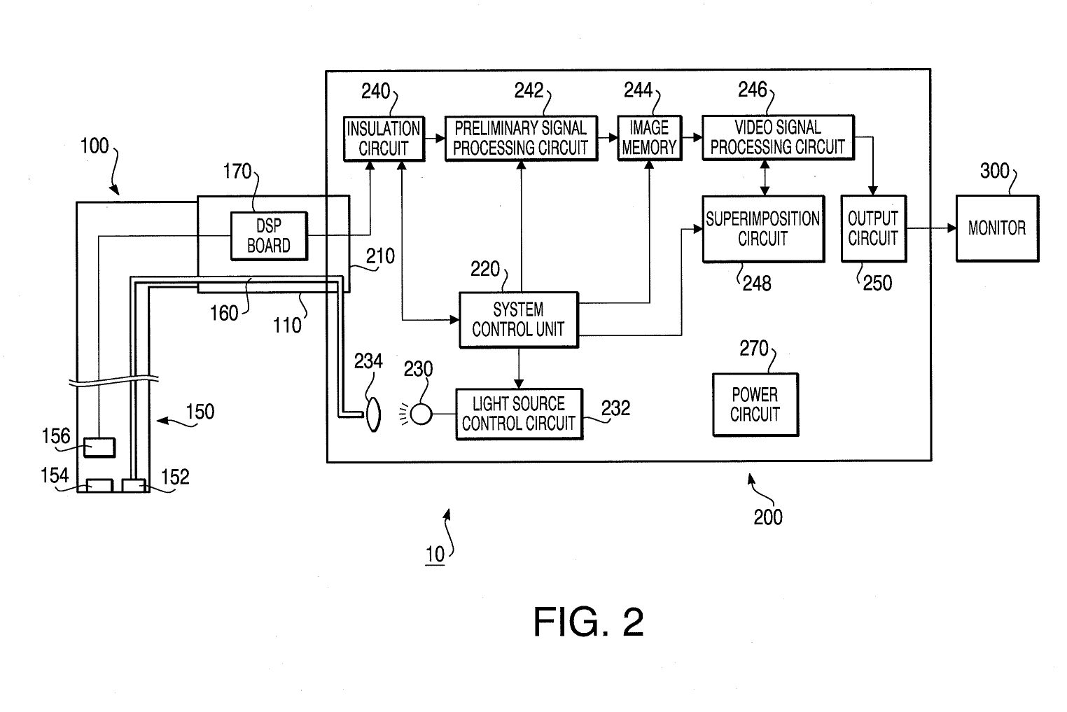

FIG. 1 is a schematic view of an endoscope system 10 according to the embodiment of the present invention. FIG. 2 is a block diagram illustrating a configuration of the endoscope system 10 according to the embodiment of the present invention.

The endoscope system 10 includes an electronic endoscope 100, a processor 200, and a monitor 300. The endoscope 100 is provided with a connector unit 100 at a distal end thereof, and the connector unit 100 has a two-prong plug 110a, which is connectable with a connector 210 of the processor 200. The connector 210 has two receive portions 210a, each of which corresponds to one of the two prongs in the two-prong plug 110a. One of the two prongs 110a and a corresponding receive portion 210a provide electrical connection between the electronic endoscope 100 and the processor unit 200, while the other of the two prongs 110a and the corresponding receive portion 210a provide optical connection.

The connector unit 110 is connected with one end of a flexible cord 120, and the other end of the cord 120 is connected with an operation unit 130. The operation unit 130 receives an operation from a user to manipulate the electronic endoscope 100. As the user handles the operation unit 130, for example, by pressing a button (not shown) on the operation unit 130, fluid such as air and cleaning liquid can be insufflated in a body cavity. The operation unit 130 is connected with one end of a flexible tube 140.

The flexible tube 140 is to be inserted in a body cavity and is provided with a front end unit 150 at a distal end thereof. As the user operates the operation unit 130 to bend the flexible tube 140 at a portion where the front end unit 150 is connected with the flexible tube 140, the front end unit 150 is angled, and an area to be observed transitions accordingly.

The front end unit 150 is formed with a hard material such as resin and provided with components required for image capturing process, which are a light distributing lens 152, an objective lens 154, and a CCD 156. The light distributing lens 152 and the objective lens 154 are arranged on a front end surface of the front end unit 150. The CCD 156 is a known color CCD in an arrangement such as Bayer arrangement.

The electronic endoscope 100 is further provided with a light guide 160, which is arranged in a longitudinal direction thereof. The light guide 160 is a bundle of optical fibers, of which one end is connected to one of the prongs of the two-prong plug in the connector unit 110. The other end of the light guide 160 is arranged in vicinity of the light distributing lens 152. The connector unit 110 is provided with a circuit board having a DSP board 170, which controls the CCD 156 and processes output signals from the CCD 156.

The processor 200 is provided with a power circuit 270, which converts commercial power source into DC power. The DC power converted in the power circuit 270 is supplied to each component in the processor 200. In FIG. 1, connection between the power circuit 270 and each component in the processor 200 is omitted for explanatory simplicity.

The processor 200 includes a system control unit 220, which controls each component in the entire endoscope system 10. Further, the processor 200 includes a light source 230 to irradiate inside the body cavity, a light source control circuit 232 to control the light source 230, and a condenser lens 234. In the present embodiment, a known white light source, such as a metal halide lamp, a xenon lamp, and a halogen lamp, is used as the light source 230. The light emitted from the light source 230 is collected by the condenser lens 234 which is arranged on a front side of the light source 230 and enters the electronic endoscope 100 (more specifically, a core of the light guide 160) through a connector portion 210 of the processor 200. The light is thus transmitted through the light guide 160 and emitted from the front end thereof through the light distributing lens 152 to irradiate the body cavity.

The irradiated light is reflected in the body cavity and enters the objective lens 154. The CCD 156 is substantially arranged in a position in which an image through the objective lens 154 is formed so that the light entering the objective lens 154 is focused on the light receiving surface of the CCD 156.

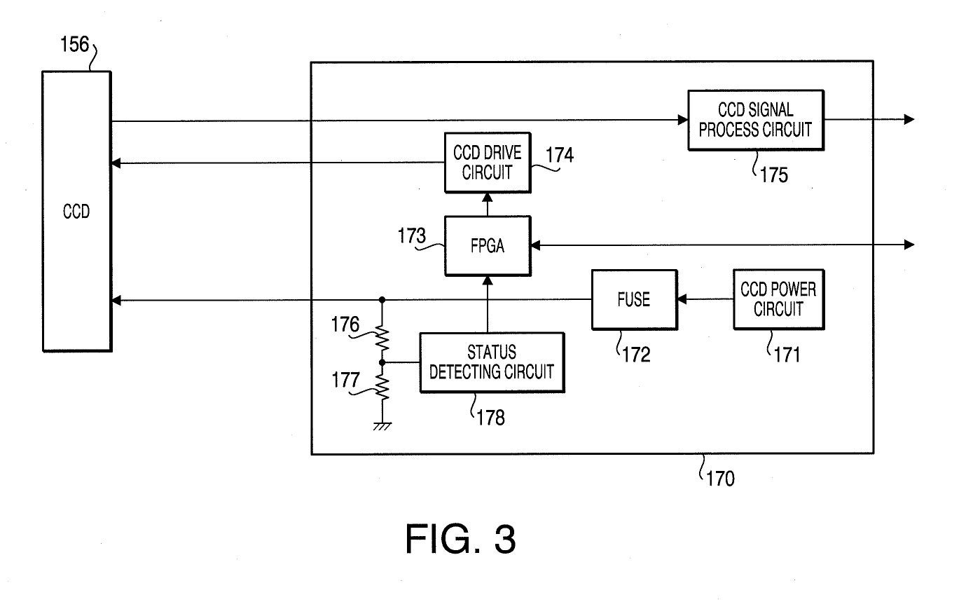

FIG. 3 is a block diagram illustrating a configuration of the CCD 156 and the DSP board 170 of the endoscope system 10 according to the embodiment of the present invention. The DSP board 170 includes a CCD power circuit 171, a fuse 172, an FPGA (field programmable gate array) 173, a CCD drive circuit 174, a CCD signal process circuit 175, resistors 176, 177, and a status detecting circuit 178 to detect status of the fuse 172.

As the DC power is supplied by the power circuit 270, the CCD power circuit 171 converts the voltages (DC-to-DC conversion) and supplies the CCD 156 with the driving voltage. The DC voltage from the power circuit 270 is also supplied to the other components in the DSP board 170.

The FPGA 173 generates control signals to drive the CCD 156 based on synchronizing pulses, which are transmitted from the system control unit 220 of the processor 200, and outputs the generated control signals to the CCD drive circuit 174. The CCD drive circuit 174 outputs drive signals to drive the CCD 156 according to the control signals.

The CCD 156 driven according to the control signals converts the optical image focused on the light receiving surface into image signals (CCD output signals) and outputs to the DSP board 170. Then, the CCD signal processing circuit 175 in the DSP board 170 generates image signals including color component signals and brightness signals based on the CCD output signals. The image signals are transmitted to the processor 200.

Referring back to FIG. 2, signal processing performed in the processor 200 will be described. The processor 200 in the present embodiment includes an insulation circuit 240, a preliminary signal processing circuit 242, an image memory 244, a video signal processing circuit 246, a superimposition circuit 248, and an output circuit 250. The insulation circuit 240 converts the signals transmitted from the electronic endoscope 100 into another transmitting signals such as optical signals by using, for example, a photo-coupler so that the electronic endoscope 100 and the processor 200 are electrically insulated at the insulation circuit 240.

The image signals output from the electronic endoscope 100 are inputted through the connector portion 210 and the insulation circuit 240 into the preliminary signal process circuit 242. The preliminary signal process circuit 242 amplifies and converts the inputted image signals (analog-to-digital conversion), and the output digital image signals are stored in the memory 244 on a frame basis. The stored image signals are output to the video signal process circuit 246 frame by frame based on timing determined with respect to the synchronizing pulses provided from the system control unit 220. The synchronizing pulses are provided to the FPGA 173 as well in substantially equal timing. Thus, the signal processing in the processor 200 and drive timing of the CCD 156 are synchronized.

The image signals are thereafter converted into signals adaptable to the monitor 300 (i.e., color component signals and brightness signals) in the video signal process circuit 246. The converted signals are further output to the output circuit 250.

The superimposition circuit 248 processes a predetermined image to be superimposed on the images captured by the electronic endoscope 100 under control of the system control unit 220 in cooperation with the video signal process circuit 246.

The output circuit 250 converts the color component signals and the brightness signals transmitted from the video signal processing circuit 246 into video signals in a predetermined format (for example, composite video signals, S-video signals, and RGB video signals) to output to the monitor 300 to be displayed. Thus, the image captured by the electronic endoscope 100 is displayed in the monitor 300 with or without the superimposed image.

With the above configuration, when one of the signal cables, for example, a cable connecting the CCD 156 and the DSP board 170 is disconnected or exposed to cause short-circuiting between a cable supplying electric power to the CCD 156 and the ground, overcurrent is caused between the CCD power circuit 171 and the CCD 156. In order to prevent the overcurrent, the fuse 172 arranged between the CCD power circuit 171 and the electric current for the CCD 156 is shutdown.

The status of the fuse is detected by the status detecting circuit 178, which monitors potential between the resistor 176 and the resistor 177. More specifically, in normal status, when the electric power is supplied to the CCD 156, the voltage is applied to the resistors 176, 177. In this state, resistance ratio of the resistor 176, 177 is set such that the potential between the resistor 176 and the resistor 177 is higher than a predetermined threshold. Thus, as the status detecting circuit 178 in the normal state detects the potential between the resistor 176 and the resistor 177 higher than the predetermined threshold, signals indicating the higher potential (hereinafter referred to as H signals) are transmitted to the FPGA 173.

The FPGA 173, which receives the H signals, determines that the fuse 172 is maintained, and the power is normally supplied to generate and output the control signals based on the synchronizing pulses.

When the overcurrent is caused, and the fuse 172 is blown to cease supplying the power to the CCD 156, the resistor 176 becomes open, and the resistor 177 serves as a pull-down resistor to lower the potential between the resistor 176 and the resistor 177 than the predetermined threshold. As the status detecting circuit 178 detects the lowered potential, L signals indicating the lower potential are transmitted to the FPGA 173.

The FPGA 173, which receives the L signals, determines that the fuse 172 is blown due to the overcurrent between the CCD 156 and the CCD power circuit 171 and the electric power is shutdown. The FPGA 173 thereafter transmits reporting signals to report the power status to the system control unit 220. Subsequently, the FPGA 173 transmits signals indicating to cease generating the control signals. Alternatively, signals indicating to cease drive signals for the CCD 156 are transmitted to the CCD drive circuit 174. Accordingly, drive signals for the CCD 156 are ceased. Therefore, drive signals for the CCD 156 are not inputted in the CCD 156 in connection with the electric power to the CCD 156 being shutdown. Thus, considerably higher voltage than the power supply voltage (e.g., 0 volt) is prevented from being applied to the CCD 156, and a latch-up phenomenon, in which the CCD 156 can be overheated and fail, can be avoided.

Further, the system control unit 220 controls the superimposition circuit 248 according to the reporting signals transmitted from the FPGA 173. The superimposition circuit 248 functions in connection with the video signal process circuit 246 to display an image indicating the failure (i.e., short-circuiting) in the electronic endoscope 100 over the image obtained through the electronic endoscope 100. Thus, the operator viewing the monitor 50 can recognize the failure in the electronic endoscope 100. It is noted that the superimposed image can be displayed partially or substantially entirely over the image obtained through the electronic endoscope 100 as long as the superimposed image is recognizable to the operator.

In the present embodiment, the fuse 172 is configured to be self-recoverable. That is, when the cause of the short-circuiting is removed, the CCD 156 and the DSP board 170 can operate normally. However, the fuse 172 may not necessarily be self-recoverable. In such a configuration, the fuse 172 requires to be exchanged for example when the electronic endoscope 100 is repaired.

It is noted that, in the power supply unit in the above-referenced publication, the overcurrent in the power supply line is monitored based on electronic current values. If the monitoring system is applied to the endoscope system of the present embodiment, the electric current value to the CCD 156 is monitored. However, in this configuration, individual specificity in, for example, the power supply line, the CCD 156, and a resistor for monitoring may affect the electric current value to be monitored. On the contrary, in the endoscope system of the present embodiment, the status of the fuse 172 is monitored to detect overcurrent. It is noted that the status of the fuse 172 is rather independent from the individual specificity of each component. Therefore, the overcurrent can be reliably detected.

Although an example of carrying out the invention has been described above, the present invention is not limited to the above described embodiment. For example, the CCD signal process circuit 175 in the DSP board 170 may be arranged in the processor 200. In this configuration, the CCD signal process circuit 175 is positioned between the insulation circuit 240 and the preliminary signal processing circuit 242. Further, the status detecting circuit 178 may be omitted when the resistance values of the resistors 176, 177 are properly adjusted, and the FPGA 173 is configured to directly detect the status of the fuse 172.

The present disclosure relates to the subject matter contained in Japanese Patent Application No. P2006-205705, filed on Jul. 28, 2006, which is expressly incorporated herein by reference in its entirety.

What is claimed is:

1. An endoscope to obtain an image inside a body cavity, comprising:
an image capturing element;
a power source, which supplies electric power to the image capturing element;
a power shutdown unit, which shuts down the electric power to the image capturing element so that the image capturing element can be prevented from overcurrent;
a drive control unit, which outputs control signals to the image capturing element in order to control driving of the image capturing element based on driving signals provided from an external unit connected with the endoscope; and
a control restricting unit, which restricts the drive control unit to cease outputting the control signals to the image capturing element in response to activation of the power shutdown unit.

2. The endoscope according to claim 1,
wherein the power shutdown unit is a fuse provided between the power source and the image capturing element, and
wherein activating the power shutdown unit is blowing the fuse.

3. The endoscope according to claim 2, further comprising:
a fuse status judging system, which judges as to whether the fuse is blown,
wherein the control restricting unit restricts the drive control unit to cease outputting the control signals to the image capturing element when the fuse status judging system judges that the fuse is blown.

4. An endoscope system, comprising:
an endoscope to obtain an image inside a body cavity;
a monitor, which displays the image obtained by the endoscope; and
a processor, which processes the image obtained by the endoscope into a format adaptable to the monitor;
wherein the endoscope includes:
an image capturing element;
a power source, which supplies electric power to the image capturing element;
a power shutdown unit, which shuts down the electric power to the image capturing element so that the image capturing element can be prevented from overcurrent;
a drive control unit, which outputs control signals to the image capturing element in order to control driving of the image capturing element based on driving signals provided from an external unit connected with the endoscope; and
a control restricting unit, which restricts the drive control unit to cease outputting the control signals to the image capturing element in response to activation of the power shutdown unit.

5. The endoscope system according to claim 4,
wherein the power shutdown unit of the endoscope is a fuse provided between the power source and the image capturing element, and
wherein activating the power shutdown unit is blowing the fuse.

6. The endoscope system according to claim 5,
wherein the endoscope further includes a fuse status judging system, which judges as to whether the fuse is blown,
wherein the control restricting unit of the endoscope restricts the drive control unit to cease outputting the control signals to the image capturing element when the fuse status judging system judges that the fuse is blown.

7. The endoscope system according to claim 4,
wherein a reporting signal to report the activation of the power shutdown unit is transmitted to the processor in response to the activation of the power shutdown unit; and
wherein the processor adds a superimposed image over the image obtained by the endoscope on the monitor in response to the reporting signal.

8. An electric power unit for an endoscope with an image capturing element to obtain an image inside a body cavity, comprising:
a power source, which supplies electric power to the image capturing element;
a power shutdown unit, which shuts down the electric power to the image capturing element so that the image capturing element can be prevented from overcurrent;
a drive control unit, which outputs control signals to the image capturing element in order to control driving of the image capturing element based on driving signals provided from an external unit connected with the endoscope; and
a control restricting unit, which restricts the drive control unit to cease outputting the control signals to the image capturing element in response to activation of the power shutdown unit.
